(19) United States
(12) Patent Application Publication
(10) Pub. No.: US 2011/0143426 A1
(43) Pub. Date: Jun. 16, 2011

(54) DEVICE AND PROCESS TO GENERATE CO2 USED FOR INDOOR CROP PRODUCTION AND UNDERWATER GARDENING

(76) Inventors: Lewis Busby, Avondale, PA (US); Zachary Busby, Avondale, PA (US); James E. Davidson, Landenberg, PA (US)

(21) Appl. No.: 13/031,652

(22) Filed: Feb. 22, 2011

Related U.S. Application Data
(62) Division of application No. 12/064,182, filed on Feb. 19, 2008.
(60) Provisional application No. 60/710,319, filed on Aug. 22, 2005.

(51) Int. Cl. C12M 1/04; C12M 1/06

(52) U.S. Cl. 435/290.1; 435/289.1

(57) ABSTRACT

A process and device used to produce CO₂ in an indoor environment is described. The device contains a container that contains a mixture of substrate and fungi. The container has at least one opening to permit the CO₂ to enter the plant growth environment and a pump connected to the container to pump out the CO₂ through said opening into the plant growth environment The invention further relates to a kit that contains a container, substrate, fungi and a pump.

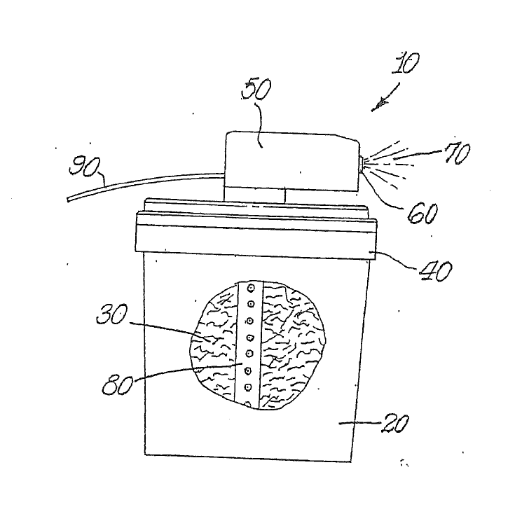

### RELATED APPLICATIONS

This application claims benefit to provisional application Ser. No. 60/710,319 that was filed Aug. 22, 2005 and is incorporated by reference in its entirety for all useful purposes.

### BACKGROUND OF THE INVENTION

Carbon dioxide is important to all living things. The reduction of carbon dioxide, through photosynthesis in the chloroplasts of green plants, results in the formation of carbohydrate, which furnishes the immediate energy needs of all plants and animals. Each growing season, tremendous quantities of atmospheric carbon dioxide diffuse into green plants, making photosynthesis possible. At the end of a season or at the end of a plant's life, carbon must be recycled back into the atmosphere so that the next generation of green plants may grow and develop. A continuous recycling of carbon is essential to life on earth.

Many processes are responsible for the return of carbon dioxide to the atmosphere including: respiration of animals, volcanic eruptions, and combustion of coal, gas, petroleum, and plants and animals. However, fungi (and bacteria) undoubtedly play the major roles in the carbon cycle. They are responsible, through their enzymatic action, for converting the carbohydrates and other carbon containing compounds back into carbon dioxide that green plants can then again synthesize to carbohydrate. This cycle has existed for millions of years on Earth and has resulted in a relatively stable atmospheric concentration of carbon dioxide and oxygen.

Fungi are eukaryotic, spore-producing, non-photosynthetic organisms that must absorb nutrients from organic matter formed by other organisms. Fungi may be unicellular (yeasts) or multicellular (mushrooms) and their cell walls usually contain chitin or cellulose and beta-glucan. The kingdom fungi offers enormous biodiversity with over 70,000 known genera and 1.5 million species.

Fungi have contributed to the shaping of humankind's welfare since the beginning of civilization. Fungi are recognized as both beneficial and harmful in their relationship to human affairs although this role is predominantly beneficial. Undoubtedly, the most important roles for fungi on earth are agents of decay. In forest ecosystems, fungi are the principal agents that decompose cellulose, hemicellulose and lignin, the primary components of wood. Fungi utilize many different substrates as food including many foodstuffs we use. As a group, the fungi have the ability to use almost any carbon source as food. The type of substrate a specific species can use for food is determined to a large extent by the type of digestive enzymes it produces and releases into its environment. The capability of fungi (and bacteria) to decay organic material through digestive and respiratory processes benefits man by: 1) removing organic debris from man's environment, 2) the formation of humus, an important soil constituent, and 3) releasing large quantities of carbon dioxide (important to photosynthesis) to the atmosphere. Carbon dioxide is routinely used to enrich or “fertilize” the environment in greenhouses or indoor production areas to enhance photosynthesis and increase commercial crop yields (Chalabi, Z. A., A. Biro, B. J. Bailey, D. P. Aikman and K. E. Cockshull. 2002, Optimal control strategies for carbon dioxide enrichment in greenhouse tomato crops—Part 1: using pure carbon dioxide, Biosystems Engineering 81:421-431; Hand, D. W. 1982.CO₂ enrichment, the benefits and problems. Scientia Horticulture 33:14-43 and Hand, D. W. 1984. Crop responses to winter and summer CO₂ enrichment. Acta Horticulturae 162:45-60). Two primary methods to fertilize indoor environments with carbon dioxide is to use pure carbon dioxide from storage tanks or to burn fossil fuels such as natural gas, coal, fuel oil, etc. These two methods have some disadvantages such has the difficulty in handling and transporting heavy carbon dioxide storage tanks and the generation of heat in the plant production environment through burning of fossil fuels. Our invention overcomes these disadvantages by providing easy to handle containers and by producing carbon dioxide through the growth of fungi in a substrate without the generation of excessive heat.

### BRIEF SUMMARY OF THE INVENTION

We describe a process and a device to generate a natural, preferably 100% organic CO₂ product for the purpose of air fertilization. Air fertilization involves increasing the levels of CO₂ in ambient air above normal levels (about 300-about 600 parts per million (ppm)) therefore enhancing the process of photosynthesis and overall plant growth. The process is useful for indoor crop production environments such as greenhouses, aquariums, garages, etc. The invention also is useful for outdoor applications for producing CO₂ such as but not limited to ponds, breeding fish, etc.

The process and device are useful for improving the growth rates and overall robustness of plants, such as but not limited to mums, geraniums, orchids, African violets, roses, begonias, basil or vegetables such as, but not limited to tomatoes, peppers, lettuce, carrots, celery, lettuce, etc.

Inside a closed greenhouse/grow room, CO₂ is generated at night through plant decaying matter in the soil. This level of CO₂ is quickly used up in the early growing hours of green plants. This cycle is obviously counterproductive in the use of daily available sunlight or the “lights on” phase of a grow room. We believe that a device according to the invention will:

Increase plant yields 50-100 percent,

Provide shorter growing periods,

Improve floral quality and health,

Reduce heating costs (less ventilation, faster growing periods),

Provide an all-natural, affordable alternative for CO₂ production.

This process and device will enrich CO₂ levels in indoor crop production environments that will enhance plant growth and robustness.

The formulation is comprised of a mixture of at least one substrate and at least one fungus. The substrate is preferably natural by-products from the farming industry (substrate). The fungi feed on the substrate and thereby release CO₂. The substrate can be considered food for the fungi.

One aspect of the invention is a device used to produce CO₂ in an indoor environment that comprises a container that contains a mixture of substrate and fungi and said container has at least one opening to permit air to enter the container and a pump connected to the container to pump out the CO₂ through said opening into the environment.

Another aspect of the invention is a process to produce CO₂ in an indoor growing environment that comprises mixing at least one substrate and at least one fungus in a container that has at least one opening to permit the CO₂ to exit the container and enter the growing environment and placing said container in an indoor growing environment.

A still further aspect of the invention is a kit that comprises a container, a pump, a substrate and fungi or bacteria.

### SUMMARY OF THE FIGURES

The figure illustrates the device according to the invention.

### DETAILED DESCRIPTION OF THE INVENTION

The invention is directed to a process and a device to generate a CO₂ product for the purpose of air fertilization. The process and the device require mixing at least one fungus with a substrate. The invention preferably relates to a kit that comprises a container, a pump, a substrate and a fungus.

The substrate to be used will include at least one of the following: poultry manure, cottonseed meal, cottonseed hulls, soybean meal, brewer's grain, coco beans shells, straw-bedded horse manure, hay, wheat straw, gypsum, wood and wood products (sawdust, woodchips, etc.), corn cobs, wheat bran, millet, rye, and other plant or animal material and other materials known by those skilled. in the art, or combinations of these materials.

The various substrates appropriate to facilitate the growth of a particular fungus are known in the art. Preferably, the species of the fungi to be used will include mycelium such as but not limited to Agaricus bisporus (button mushroom) and Pleurotus spp. (oyster mushrooms).

Because the fungi share characteristics of both plants and animals, they are classified separately in the Kingdom Fungi. Within this Kingdom, there are the “filamentous fungi,” so named because their vegetative bodies consist of small filaments referred to as “hyphae.” Typically, the hyphae grow in a branching fashion, spreading over or within the substrate used as the source of nourishment, thereby forming a network of hyphae called “mycelium.” In the life cycle of most filamentous fungi, the mycelium gives rise to either asexual or sexual reproductive bodies bearing spores. The spore is functionally comparable to the seed of higher plants, being important in the dispersal and survival of the fungus in nature. Under suitable environmental conditions, the spore germinates to form another generation of hyphae and, thus, completes the life cycle of the fungus.

The by-products will be environmentally conditioned to produce a carbohydrate-containing substrate. Conditioning the substrate involves temperature manipulation, introducing fresh ambient air into a controlled growing environment, pasteurizing or sterilizing the substrate and introducing protein-rich supplements such as dried blood, corn meal, delayed release nutrients, etc, into the substrate. The combination of these materials and their subsequent maturation will initiate an organic reaction that releases naturally produced CO₂ into the growing environment therefore increasing the process of photosynthesis. Once fungi are introduced and start to grow in the substrate they will begin to consume the carbohydrate-containing substrate, start producing CO₂ and increase the rate of photosynthesis and subsequent plant growth.

Photosynthesis is defined as the process of converting radiant energy (sunlight, etc), H₂O, and CO₂ into oxygen that is released to the atmosphere and into carbohydrates and other organic substances that are stored energy sources in plants.

The figure illustrates one embodiment according to the invention. The device 10 is shown in the figure. The substrate and fungi mixture 30 can be placed in a container 20 specifically designed to maximize the release of natural CO₂ into various growing environments. The container 20 will be designed to encase the substrate and fungi mixture 30. The container will be equipped with at least one venting hole. The venting hole can be in the lid 40 to facilitate the release of naturally produced CO₂.

A pump system 50 can also be included to assist in the extraction of CO₂ from the containers. Any known pump 50 can be used. In the figure, the pump 50 is an electrical pump (with the electrical cord 90 shown in the Figure). The pump 50 can have a tube 80 going into the fungi and substrate mixture 30. The CO₂ 70 that is produced could travel from the tube 80 and get pumped out of the container 20 though such means as a nozzle 60. It is preferable to match the air pump with the size of the container. If a one-gallon container is used, it is preferable to use an aquarium air pump. If a 55-gallon drum is used, the pump should be a much larger pump.

The container 20 is preferably plastic or metal and most preferably plastic. The size of the container does not matter but it is easier to work with one or 5-gallon containers.

In addition, the apparatus to produce CO₂ may contain an agitator (although not shown) that may be located in a similar location to the tube 80. The agitator will enhance the amount of CO₂ produced by breaking up the substrate and stimulating the fungi to feed on the substrate. An agitator can be any known agitator as long as it can break up the substrate and fungi mixture. The purpose of the agitator is to perturb the mycelium so that additional growth will occur. Examples of an agitator include any type of stirrer, vibrator, orbital sander, etc.

Mushroom farming consists of six steps, and although the divisions are somewhat arbitrary, these steps identify what is needed to form a production system. This is described in detail in http://www.mushroominfo.com/grow/sixsteps.html that is incorporated by reference in its entirety for all useful purposes. This reference states the following:

One of the preferred embodiments is to use Phase II compost made as described below:

The six steps of mushroom farming are Phase I composting, Phase II composting, spawning, casing, pinning, and cropping. These steps are described in their naturally occurring sequence, emphasizing the salient features within each step. Compost provides nutrients needed for mushrooms to grow. Two types of material are generally used for mushroom compost, the most used and least expensive being wheat straw-bedded horse manure. Synthetic compost is usually made from hay and crushed corncobs, although the term often refers to any mushroom compost where the prime ingredient is not horse manure. Both types of compost require the addition of nitrogen supplements and a conditioning agent, gypsum.

The preparation of compost occurs in two steps referred to as Phase I and Phase II composting. The discussion of compost preparation and mushroom production begins with Phase I composting.

Phase I: Making Mushroom Compost

This phase of compost preparation usually occurs outdoors although an enclosed building or a structure with a roof over it may be used. A concrete slab, referred to as a wharf, is required for composting. In addition, a compost turner to aerate and water the ingredients, and a tractor-loader to move the ingredients to the turner is needed. In earlier days piles were turned by hand using pitchforks, which is still an alternative to mechanized equipment, but it is labor intensive and physically demanding.

Phase I composting is initiated by mixing and wetting the ingredients as they are stacked in a rectangular pile with tight sides and a loose center. Normally, the bulk ingredients are put through a compost turner. Water is sprayed onto the horse manure or synthetic compost as these materials move through the turner. Nitrogen supplements and gypsum are spread over the top of the bulk ingredients and are thoroughly mixed by the turner. Once the pile is wetted and formed, aerobic fermentation (composting) commences as a result of the growth and reproduction of microorganisms, which occur naturally in the bulk ingredients. Heat, ammonia, and carbon dioxide are released as by-products during this process. Compost activators, other than those mentioned, are not needed, although some organic farming books stress the need for an “activator.”

Mushroom compost develops as the chemical nature of the raw ingredients is converted by the activity of microorganisms, heat, and some heat-releasing chemical reactions. These events result in a food source most suited for the growth of the mushroom to the exclusion of other fungi and bacteria. There must be adequate moisture, oxygen, nitrogen, and carbohydrates present throughout the process, or else the process will stop. This is why water and supplements are added periodically, and the compost pile is aerated as it moves through the turner.

Gypsum is added to minimize the greasiness compost normally tends to have. Gypsum increases the flocculation of certain chemicals in the compost, and they adhere to straw or hay rather than filling the pores (holes) between the straws. A side benefit of this phenomenon is that air can permeate the pile more readily, and air is essential to the composting process. The exclusion of air results in an airless (anaerobic) environment in which deleterious chemical compounds are formed which detract from the selectivity of mushroom compost for growing mushrooms. Gypsum is added at the outset of composting at 40 lbs. per ton of dry ingredients.

Nitrogen supplements in general use today includes brewer's grain, seed meals of soybeans, peanuts, or cotton, and chicken manure, among others. The purpose of these supplements is to increase the nitrogen content to 1.5 percent for horse manure or 1.7 percent for synthetic, both computed on a dry weight basis. Synthetic compost requires the addition of ammonium nitrate or urea at the outset of composting to provide the compost microflora with a readily available form of nitrogen for their growth and reproduction.

Corncobs are sometimes unavailable or available at a price considered to be excessive. Substitutes for or complements to corncobs include shredded hardwood bark, cottonseed hulls, neutralized grape pomace, and cocoa bean hulls. Management of a compost pile containing any one of these materials is unique in the requirements for watering and the interval between turnings.

The initial compost pile should be 5 to 6 feet wide, 5 to 6 feet high, and as long as necessary. A two-sided box can be used to form the pile (rick), although some turners are equipped with a “ricker” so a box isn't needed. The sides of the pile should be firm and dense, yet the center must remain loose throughout Phase I composting. As the straw or hay softens during composting, the materials become less rigid and compactions can easily occur. If the materials become too compact, air cannot move through the pile and an anaerobic environment will develop.

Turning and watering are done at approximately 2-day intervals, but not unless the pile is hot (145° to 170° F.). Turning provides the opportunity to water, aerate, and mix the ingredients, as well as to relocate the straw or hay from a cooler to a warmer area in the pile, outside versus inside. Supplements are also added when the ricks are turned, but they should be added early in the composting process. The number of turnings and the time between turnings depends on the condition of the starting material and the time necessary for the compost to heat to temperatures above 145° F.

Water addition is critical since too much will exclude oxygen by occupying the pore space, and too little can limit the growth of bacteria and fungi. As a general rule, water is added up to the point of leaching when the pile is formed and at the time of first turning, and thereafter either none or only a little is added for the duration of composting. On the last turning before Phase LI composting, water can be applied generously so that when the compost is tightly squeezed, water drips from it. There is a link between water, nutritive value, microbial activity, and temperature, and because it is a chain, when one condition is limiting for one factor, the whole chain will cease to function. Biologists see this phenomenon repeatedly and have termed it the Law of Limiting Factors.

Phase I composting lasts from 7 to 14 days, depending on the nature of the material at the start and its characteristics at each turn. There is a strong ammonia odor associated with composting, which is usually complemented by a sweet, moldy smell. When compost temperatures are 155° F. and higher, and ammonia is present, chemical changes occur which result in a food rather exclusively used by the mushrooms. As a by-product of the chemical changes, heat is released and the compost temperatures increase. Temperatures in the compost can reach 170° to 180° F. during the second and third turnings when a desirable level of biological and chemical activity is occurring. At the end of Phase I the compost should: a) have a chocolate brown color; b) have soft, pliable straws, c) have a moisture content of from 68 to 74 percent; and d) have a strong smell of ammonia. When the moisture, temperature, color, and odor described have been reached, Phase I composting is completed.

Phase II: Finishing the Compost

There are two major purposes to Phase II composting. Pasteurization is necessary to kill any insects, nematodes, pest fungi, or other pests that may be present in the compost. And second, it is necessary to remove the ammonia that formed during Phase I composting. Ammonia at the end of Phase II in a concentration higher than 0.07 percent is often lethal to mushroom spawn growth, thus it must be removed; generally, a person can smell ammonia when the concentration is above 0.10 percent.

Phase II takes place in one of three places, depending on the type of production system used. For the zoned system, compost is packed into wooden trays, the trays are stacked six to eight high, and are moved into an environmentally controlled Phase II room. Thereafter, the trays are moved to special rooms, each designed to provide the optimum environment for each step of the mushroom growing process. With a bed or shelf system, the compost is placed directly in the beds, which are in the room used for all steps of the crop culture. The most recently introduced system, the bulk system, is one in which the compost is placed in a cement-block bin with a perforated floor and no cover on top of the compost; this is a room specifically designed for Phase II composting.

The compost, whether placed in beds, trays, or bulk, should be filled uniformly in depth and density or compression. Compost density should allow for gas exchange, since ammonia and carbon dioxide will be replaced by outside air.

Phase II composting can be viewed as a controlled, temperature-dependent, ecological process using air to maintain the compost in a temperature range best suited for the de-ammonifying organisms to grow and reproduce. The growth of these thermophilic (heat-loving) organisms depends on the availability of usable carbohydrates and nitrogen, some of the nitrogen in the form of ammonia.

Optimum management for Phase II is difficult to define and most commercial growers tend toward one of the two systems in general use today: high temperature or low temperature.

A high temperature Phase II system involves an initial pasteurization period during which the compost and the air temperature are raised to at least 145° F. for 6 hours. Heat generated during the growth of naturally occurring microorganisms or by injecting steam into the room where the compost has been placed, or both can accomplish this. After pasteurization, the compost is re-conditioned by immediately lowering the temperature to 140° F. by flushing the room with fresh air. Thereafter, the compost is allowed to cool gradually at a rate of approximately 2° to 3° F. each day until all the ammonia is dissipated. This Phase II system requires approximately 10 to 14 days to complete.

In the low temperature Phase II system the compost temperature is initially increased to about 126° F. with steam or by the heat released via microbial growth, after which the air temperature is lowered so the compost is in a temperature range of 125° to 130° F. range. During the 4 to 5 days after pasteurization, the compost temperature may be lowered by about 2° F. a day until the ammonia is dissipated.

It is important to remember the purposes of Phase II when trying to determine the proper procedure and sequence to follow. One purpose is to remove unwanted ammonia. To this end the temperature range from 125° to 130° F. is most efficient since de-ammonifying organisms grow well in this temperature range. A second purpose of Phase II is to remove any pests present in the compost by use of a pasteurization sequence.

At the end of Phase II the compost temperature must be lowered to approximately 75° to 80° F. before spawning (planting) can begin. The nitrogen content of the compost should be 2.0 to 2.4 percent, and the moisture content between 68 and 72 percent. Also, at the end of Phase II it is desirable to have 5 to 7 lbs. of dry compost per square foot of bed or tray surface to obtain profitable mushroom yields. It is important to have both the compost and the compost temperatures uniform during the Phase II process since it is desirable to have as homogenous a material as possible.

Spawning

Mushroom compost must be inoculated with mushroom spawn (Latin expandere=to spread out) if one expects mushrooms to grow. The mushroom itself is the fruit of a plant as tomatoes are of tomato plants. Within the tomato one finds seeds, and these are used to start the next season's crop. Microscopic spores form within a mushroom cap, but their small size precludes handling them like seeds. As the tomato comes from a plant with roots, stems, and leaves, the mushroom arises from thin, thread-like cells called mycelium. Fungus mycelium is the white, thread-like plant often seen on rotting wood or moldy bread. Mycelium can be propagated vegetatively, like separating daffodil bulbs and getting more daffodil plants. Specialized facilities are required to propagate mycelium, so the mushroom mycelium does not get mixed with the mycelium of other fungi. Mycelium propagated vegetatively is known as spawn, and commercial mushroom farmers purchase spawn from any of about a dozen spawn companies.

Spawn makers start the spawn-making process by sterilizing a mixture of rye grain plus water and chalk; wheat, millet, and other small grain may be substituted for rye. Sterilized horse manure formed into blocks was used as the growth medium for spawn up to about 1940, and this was called block or brick spawn, or manure spawn; such spawn is uncommon now. Once sterilized grain has a bit of mycelium added to it, the grain and mycelium is shaken 3 times at 4-day intervals over a 14-day period of active mycelial growth. Once the grain is colonized by the mycelium, the product is called spawn. Spawn can be refrigerated for a few months, so spawn is made in advance of a farmer's order for spawn.

In the United States, mushroom growers have a choice of four major mushroom cultivars: a) Smooth white—cap smooth, cap and stalk white; b) Off-white—cap scaly with stalk and cap white; c) Cream—cap smooth to scaly with stalk white and cap white to cream; and d) Brown—cap smooth, cap chocolate brown with a white stalk. Within each of the four major groups, there are various isolates, so a grower may have a choice of up to eight smooth white strains. The isolates vary in flavor, texture, and cultural requirements, but they are all edible mushrooms.

Spawn is distributed on the compost and then thoroughly mixed into the compost. For years this was done by hand, broadcasting the spawn over the surface of the compost and ruffling it in with a small rake-like tool. In recent years, however, for the bed system, spawn is mixed into the compost by a special spawning machine that mixes the compost and spawn with tines or small finger-like devices. In a tray or batch system, spawn is mixed into the compost as it moves along a conveyer belt or while falling from a conveyor into a tray. The spawning rate is expressed as a unit or quart per so many square feet of bed surface; 1 unit per 10 ft is desirable. The rate is sometimes expressed on the basis of spawn weight versus compost weight; a 2 percent spawning rate is

Once the spawn has been mixed throughout the compost and the compost worked so the surface is level, the compost temperature is maintained at 75° F. and the relative humidity is kept high to minimize drying of the compost surface or the spawn. Under these conditions the spawn will grow—producing a thread-like network of mycelium throughout the compost. The mycelium grows in all directions from a spawn grain, and eventually the mycelium from the different spawn grains fuses together, making a spawned bed of compost one biological entity. The spawn appears as a white to blue-white mass throughout the compost after fusion has occurred. As the spawn grows it generates heat, and if the compost temperature increases to above 80° to 85° F., depending on the cultivar, the heat may kill or damage the mycelium and eliminate the possibility of maximum crop productivity and/or mushroom quality. At temperatures below 74° F., spawn growth is slowed and the time interval between spawning and harvesting is extended.

The time needed for spawn to colonize the compost depends on the spawning rate and its distribution, the compost moisture and temperature, and the nature or quality of the compost. Complete spawn run usually requires 14 to 21 days. Once the compost is fully-grown with spawn, the next step in production is at hand.

The substrate made by the steps above is preferable.

It is also preferable to transfer the substrate and fungi mixture from a smaller container to a larger container. The transfer of the mixture to a larger container allows more air to enter the container and improves the production of CO₂. The small container slows the production of CO₂ because the fungi are starving for oxygen and they are not converting the substrate as rapidly. The transfer of the mixture to a larger container is generally from at about 2 to about 52 weeks and preferably about 3 weeks to about 6 weeks.

It is preferable to connect the pump to the mixture from about 1 to about 3 weeks and even more preferably about 2 weeks after the mixture is made. Inserting the pump at a later time permits the fungi to start feeding on the substrate thereby producing more CO₂.

It is possible to use more than one of the devices according to the invention in multiple locations in an indoor growing environment. Depending on the room size, two or more devices can be used in different locations of the environment to maximize the amount of the CO₂ to be produced in the growing environment.

Placing the mixture of substrate and fungi in a one-gallon container can produce CO₂ for 60 days in a room approximately 8×8×8 (8 feet high by 8 feet wide by 8 feet deep).

If the invention is used in a very small growing area (closet, refrigerator, etc.) too much CO₂ can be a problem. However, putting the invention on a timer and releasing CO₂ for only 3-4 hours throughout the day would be one way to prevent too much CO₂ to enter the room.

Optimum temperatures for best growing conditions vary depending on the type of plant or vegetable. However, most like to be within a range from about 65 to about 75 degrees Fahrenheit. The night (lights off) temperature should be within about 5 to about 10 degrees lower than the daytime (lights on) temperature. The humidity should preferably be between about 70-75%.

For best growing results, levels of CO₂ should be kept between 800-1100 PPM. Research has shown that the maximum growth rate for many plant species is achieved at about 1100 PPM; however, concentrations of CO₂ higher than 1800 PPM may actually slow down the growth process.

All the references described above are incorporated by reference in its entirety for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

1-22. (canceled)

23. A device used to produce CO₂ in an indoor environment comprising a container, a mixture of substrate and fungi in said container, at least one opening in said container extending from within said container to the outside of said container, and said at least one opening comprising structure for air and CO₂ to enter/exit said container.

24. The device of claim 23 including a pump connected to said container to pump the CO₂ through said at least one opening and into the environment.

25. The device of claim 24 including a tube connected to said pump and extending into said mixture for conveying the CO₂ out of said container.

26. The device of claim 24 wherein said pump is an aquarium pump.

27. The device of claim 23 including an agitator extending into said mixture to enhance the amount of CO₂ being produced by said mixture.

28. The device of claim 23 wherein said mixture is fungi and compost.

29. The device of claim 23 wherein the CO₂ is discharged directly from said at least one opening to the atmosphere surrounding said container.

30. The device of claim 23 wherein said substrate is natural by-products from the farming industry, and said fungi containing mycelium.

31. The device of claim 23 wherein said mixture comprises fungi and Phase II compost.

32. The device of claim 23 wherein said substrate is a conditioned substrate.

33. The device of claim 23 wherein said container is not air-tight.

34. The device of claim 23, in combination with said indoor environment, and said indoor environment having plant growth.

35. The device of claim 34 wherein said at least one opening permits the CO₂ to exit from said container directly into said indoor environment without passing through any intervening media.

36. The device of claim 34 wherein said container is located in said indoor environment.

37. The device of claim 24 wherein said container and said mixture and said pump are part of a kit.
